(19) United States
(12) Patent Application Publication
(10) Pub. No.: US 2023/0243849 A1
(43) Pub. Date: Aug. 3, 2023

(54) Identifying Risk of Cerebra Edema

(71) Applicant: University of Kentucky Research Foundation, Lexington, KY (US)

(72) Inventors: Keith R. Pennypacker, Lexington, KY (US); Justin F. Fraser, Lexington, KY (US); Qiang Cheng, Lexington, KY (US)

(21) Appl. No.: 17/927,389

(22) PCT Filed: May 26, 2021
(86) PCT No.: PCT/US2021/034279

§ 371 Date: Nov. 23, 2022

Related U.S. Application Data
(60) Provisional application No. 63/030,259, filed on May 26, 2020.

(51) Int. Cl. G01N 33/68

(52) U.S. Cl. CPC G01N 33/6893; G01N 2333/7155; G01N 2800/50; G01N 2333/7158; G01N 2800/2871

(57) ABSTRACT

Tools for identifying risk of cerebral edema in a stroke patient are provided and include a method that involves identifying risk of cerebral edema when cytokines and chemokines are detected in a systemic blood sample from the subject.

### RELATED APPLICATIONS

This application claims priority from U.S. Provisional Application Ser. No. 63/030,259 filed May 26, 2020, the entire disclosure of which is incorporated herein by this reference.

### TECHNICAL FIELD

The presently-disclosed subject matter generally relates to prediction of cerebral edema in a subject. In particular, certain embodiments of the presently-disclosed subject matter relate to methods and devices for use in identifying risk of cerebral edema and severe infarct volume in a subject who is a stroke patient.

### INTRODUCTION

Each year approximately 800,000 individuals have a stroke, of which 87% are ischemic.¹ Ischemic stroke remains one of the most debilitating diseases and is the fifth leading cause of death in the United States.

Emergent large vessel occlusions (ELVOs) represent 30-40% of ischemic strokes and are the most severe acute cerebral blockages. Many randomized clinical trials have demonstrated the superiority of mechanical thrombectomy (MT) over medical management alone; as a result, thrombectomy has become standard treatment for ELVO.²⁻⁵ MT involves removing the thrombus with a stent retriever to open a blocked cerebral artery. Through this technique, distal blood within the artery immediately downstream from the clot can be isolated, peripheral blood just proximal to the clot (systemic arterial blood in the cervical carotid artery), and the thrombus itself upon removal from the human subject.

From this technique, a prospective tissue bank has been established. The Blood and Clot Thrombectomy Registry and Collaboration (BACTRAC) tissue bank has been initiated to evaluate molecular mechanisms of stroke in the human condition (clinicaltrials.gov NCT03153683).⁶ The BACTRAC protocol isolates intracranial blood within the artery immediately downstream of the clot, in the clot itself, and in systemic blood proximal to the clot. Since the establishment of BACTRAC, efforts have been focused on understanding acid-base balance, electrolyte chemistry, and transcriptomics at the site of the occlusion.^{7,8}

Among the most serious conditions associated with stroke is cerebral edema, which predicts poor outcomes and even increase mortality in stroke patients. While there are a number of treatments that have been identified for cerebral edema, many have significant side effects.³²⁻³⁴ Thus, it is desirable to provide such treatment only when necessary, such as in the case of an actual occurrence of severe edema characterized by pressure in the brain endangering the life of the subject. Unfortunately, as time progresses without treatment in a subject who has an unknown risk of cerebral edema, clinical outcome declines, and can even lead to death of the subject.

Therefore, there is a critical need to be able to identify a subject who is at risk of cerebral edema, and to administer early treatment.

### SUMMARY

The presently-disclosed subject matter meets some or all of the above-identified needs, as will become evident to those of ordinary skill in the art after a study of information provided in this document.

The presently-disclosed subject matter includes methods and devices for use in identifying risk of cerebral edema and severe infarct volume in a subject. In some embodiments of the presently-disclosed subject matter a method is provided for predicting or identifying risk of cerebral edema in a subject, which involves obtaining a sample comprising systemic blood from the subject; detecting proteins in the sample, including cytokines and chemokines; and identifying risk of cerebral edema in the subject when the cytokines and chemokines are detected in the sample.

In some embodiments, a method of predicting or identifying risk of cerebral edema in a subject involves obtaining a sample comprising systemic blood from the subject; detecting REG3A, CCL18, IL20RA, and IL10RA proteins in the sample; and identifying risk of cerebral edema in the subject when REG3A, CCL18, IL20RA, and IL10RA are detected in the sample. In some embodiments, the method also involves detecting amounts of TNFRS9, ILS, KIT, TNF, CCL16, and GNLY in the samples, and predicting infarct volume when TNFRS9, ILS, KIT, TNF, CCL16, and GNLY are detected in the sample.

As noted herein, exemplary methods involve detecting proteins in a sample comprising systemic blood from the subject. In some embodiments, the method further involves isolating plasma from the sample for use in detecting the proteins. In this regard, if desired, the leukocyte component of the blood can be reserved for further testing, for example, for use in detecting mRNA (cDNA) of interest.

In connection with methods disclosed herein, the sample can be collected from a subject who is a stroke patient. For example, in some embodiments, the subject has had an ischemic stroke. In some embodiments, the subject has a medium or large volume occlusion. In some embodiments, the subject has undergone mechanical thrombectomy (MT).

As noted, in some embodiments, the subject has undergone MT. In this regard, in some embodiments, the systemic blood sample is peripheral blood collected just proximal to a thrombus or clot removed by the MT. In some embodiments, the method also involves obtaining a proximal sample comprising blood collected proximal to a thrombus in the subject, and a distal sample comprising blood collected distal to the thrombus in the subject. In some embodiments, the method also involves detecting cytokines and chemokines in the proximal sample and the distal sample, and predicting cerebral edema when there are increased amounts of cytokines and chemokines in the proximal sample as compared to the distal sample. In some embodiments, the method also involves detecting REG3A, CCL18, IL20RA, and IL10RA proteins in the proximal sample and the distal sample, and predicting cerebral edema when there are increased amounts of REG3A, CCL18, IL20RA, and IL10RA proteins in the proximal sample as compared to the distal sample.

In some embodiments, a method of predicting infarct volume in a subject involves obtaining a sample comprising systemic blood from the subject; detecting REG3A, CCL18, IL20RA, IL10RA, TNFRS9, ILS, KIT, TNF, CCL16, and GNLY proteins in the sample; and predicting infarct volume in the subject when REG3A, CCL18, IL20RA, IL10RA, TNFRS9, ILS, KIT, TNF, CCL16, and GNLY are detected in the sample.

As noted above, in some embodiments, the subject has undergone MT. In this regard, in some embodiments, the systemic blood sample is peripheral blood collected just proximal to a thrombus or clot removed by the MT. In some embodiments, the method also involves obtaining a proximal sample comprising blood collected proximal to a thrombus in the subject, and a distal sample comprising blood collected distal to the thrombus in the subject. In some embodiments, the method also involves detecting cytokines and chemokines in the proximal sample and the distal sample, and predicting cerebral edema when there are increased amounts of cytokines and chemokines in the proximal sample as compared to the distal sample. In some embodiments, the method also involves detecting REG3A, CCL18, IL20RA, IL10RA, TNFRS9, ILS, KIT, TNF, CCL16, and GNLY proteins in the proximal sample and the distal sample, and predicting cerebral edema when there are increased amounts of REG3A, CCL18, IL20RA, IL10RA, TNFRS9, ILS, KIT, TNF, CCL16, and GNLY proteins in the proximal sample as compared to the distal sample.

The presently-disclosed subject matter further includes devices for use in identifying risk of cerebral edema and severe infarct volume in a subject. In some embodiments, a device of the presently-disclosed subject matter includes a combination of probes specific for a panel of cytokines and chemokines. In some embodiments, the device includes a combination of probes including a probe specific for each of REG3A, CCL18, IL20RA, and IL10RA. In some embodiments, the device includes a combination of probes including a probe specific for each of REG3A, CCL18, IL20RA, IL10RA, TNFRS9, ILS, KIT, TNF, CCL16, and GNLY. The device can be, for example, a microfluidic enzyme-linked immunosorbent assay (ELISA) device. In some embodiments of the methods disclosed herein, the detection can be conducted using a device as disclosed herein.

In some embodiments of the presently-disclosed subject matter, when risk of cerebral edema in the subject has been identified, the method further involves administering or having administered a treatment to the subject. The administered treatment can be capable of mitigating cerebral edema. In some embodiments, the treatment includes a therapeutic agent. In some embodiments, the treatment includes surgery, such as a decompressive craniectomy.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A. Cardiometabolic panel volcano plot illustrating proteomic log 2 fold changes in Normalized Protein eXpression (NPX) in intracranial blood compared with systemic blood. Labeled proteins include prolyl endopeptidase (FAP), phospholipid transfer protein (PLTP), fetuin-B (FETUB), uromodulin (UMOD), ficolin-2 (FCN2), and superoxide dismutase 1 (SOD1).

FIG. 1B. Inflammatory panel volcano plot illustrating proteomic log 2 fold changes in Normalized Protein eXpression (NPX) in intracranial blood compared with systemic blood. Labeled proteins include C—C motif chemokine 19 (CCL19), C—C motif chemokine 20 (CCL20), fibroblast growth factor 21 (FGF21), transforming growth factor alpha (TGF-_{α}), C—C motif chemokine 23 (CCL23), and axin-1 (AXIN1).

FIGS. 2A and 2B. Predicted and measured edema values by Lasso on training data (FIG. 2A) and testing data (FIG. 2B) with a ratio of 4:1 random split. Lasso model was determined with 5-fold cross-validation.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is based in part on efforts to address the need for early identification of a subject who is at risk of cerebral edema. In a subject who has a risk of cerebral edema that is undiscovered, as time progresses without treatment, clinical outcome declines, and can even lead to death. The presently-disclosed subject matter includes methods and devices for use in identifying risk of cerebral edema and severe infarct volume in a subject, which allows for early treatment, and improved clinical outcomes.

In some embodiments of the presently-disclosed subject matter a method is provided for predicting or identifying risk of cerebral edema in a subject, which involves obtaining a sample comprising systemic blood from the subject; detecting proteins in the sample, including cytokines and chemokines; and identifying risk of cerebral edema in the subject when the cytokines and chemokines are detected in the sample.

In some embodiments, a method of predicting or identifying risk of cerebral edema in a subject involves obtaining a sample comprising systemic blood from the subject; detecting REG3A, CCL18, IL20RA, and IL10RA proteins in the sample; and identifying risk of cerebral edema in the subject when REG3A, CCL18, IL20RA, and IL10RA are detected in the sample. In some embodiments, the method also involves detecting amounts of TNFRS9, ILS, KIT, TNF, CCL16, and GNLY in the samples, and predicting infarct volume when TNFRS9, ILS, KIT, TNF, CCL16, and GNLY are detected in the sample.

Various techniques for detecting polypeptides or proteins in a sample are known to those of ordinary skill in the art, and can be used in connection with the presently-disclosed subject matter. For example, mass spectrometry and/or immunoassay devices and methods can be used, although other methods are well-known to those skilled in the art. Such devices and methods can make use of labeled molecules in various sandwich, competitive, or non-competitive assay formats, to generate a signal that is related to the presence or amount of a polypeptide of interest.

Mass spectrometry (MS) analysis can also be used alone or in combination with other methods (e.g., immunoassays) to determine the presence and/or quantity of polypeptides of interest in a biological sample. In some embodiments, the MS analysis comprises matrix-assisted laser desorption/ionization (MALDI) time-of-flight (TOF) MS analysis, such as for example direct-spot MALDI-TOF or liquid chromatography MALDI-TOF mass spectrometry analysis. Methods for utilizing MS analysis, including MALDI-TOF MS, to detect the presence and quantity of biomarker peptides in biological samples are known in the art.

Additionally, certain methods and devices, such as biosensors and optical immunoassays, can be employed to determine the presence or amount of polypeptides without the need for a labeled molecule. Is will also be known to the skilled artisan, the presence or amount of a polypeptide can be determined using a probe, such as, for example, antibodies or fragments thereof specific for each polypeptide and detecting specific binding. Any suitable immunoassay can be utilized, for example, enzyme-linked immunoassays (ELISA), radioimmunoassays (RIAs), competitive binding assays, and the like. Specific immunological binding of the antibody to the marker can be detected directly or indirectly.

The use of immobilized antibodies or fragments thereof specific for the polypeptides can be used. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on solid support. This strip can then be dipped into the test biological sample and then processed quickly through washes and detection steps to generate a measurable signal, such as for example a colored spot. A device could also include, for example, a microfluidic ELISA device.

The presently-disclosed subject matter further includes devices for use in identifying risk of cerebral edema and severe infarct volume in a subject. In some embodiments, a device of the presently-disclosed subject matter includes a combination of probes specific for a panel of cytokines and chemokines. In some embodiments, the device includes a combination of probes including a probe specific for each of REG3A, CCL18, IL20RA, and IL10RA. In some embodiments, the device includes a combination of probes including a probe specific for each of REG3A, CCL18, IL20RA, IL10RA, TNFRS9, ILS, KIT, TNF, CCL16, and GNLY. The device can be, for example, a microfluidic enzyme-linked immunosorbent assay (ELISA) device. In some embodiments of the methods disclosed herein, the detection can be conducted using a device as disclosed herein.

As noted herein, exemplary methods of the presently-disclosed subject matter involve detecting proteins in a sample that includes systemic blood from the subject. In some embodiments, the method further involves isolating plasma from the sample for use in detecting the proteins. In this regard, if desired, the leukocyte component of the blood can be reserved for further testing, for example, for use in detecting mRNA (cDNA) of interest. Techniques of isolating plasma and/or leukocytes from a blood sample are known to those of ordinary skill in the art.

In connection with methods disclosed herein, the sample can be collected from a subject who is a stroke patient. For example, in some embodiments, the subject has had an ischemic stroke. In some embodiments, the subject has a medium or large volume occlusion. In some embodiments, the subject has undergone mechanical thrombectomy (MT). As will be appreciated by the skilled artisan, certain relative terms are frequently used in the art and have specifically-understood meanings in the context of the art. In this regard, one of ordinary skill in the art would be aware of and would know what is and what is not a “medium” volume occlusion, or what is a “large” volume occlusion. Likewise, one of ordinary skill in the art would recognize terms of art such as “severe” infarct or “severe” edema, and would immediately understand what is and what is not severe.

As noted above, in some embodiments, the subject has undergone MT. In this regard, in some embodiments, the systemic blood sample is peripheral blood collected just proximal to a thrombus or clot removed by the MT. In some embodiments, the method also involves obtaining a proximal sample comprising blood collected proximal to a thrombus in the subject, and a distal sample comprising blood collected distal to the thrombus in the subject. In some embodiments, the method also involves detecting cytokines and chemokines in the proximal sample and the distal sample, and predicting cerebral edema when there are increased amounts of cytokines and chemokines in the proximal sample as compared to the distal sample. In some embodiments, the method also involves detecting REG3A, CCL18, IL20RA, and IL10RA proteins in the proximal sample and the distal sample, and predicting cerebral edema when there are increased amounts of REG3A, CCL18, IL20RA, and IL10RA proteins in the proximal sample as compared to the distal sample.

In some embodiments, a method of predicting infarct volume in a subject involves obtaining a sample comprising systemic blood from the subject; detecting REG3A, CCL18, IL20RA, IL10RA, TNFRS9, ILS, KIT, TNF, CCL16, and GNLY proteins in the sample; and predicting infarct volume in the subject when REG3A, CCL18, IL20RA, IL10RA, TNFRS9, ILS, KIT, TNF, CCL16, and GNLY are detected in the sample.

As noted above, in some embodiments, the subject has undergone MT. In this regard, in some embodiments, the systemic blood sample is peripheral blood collected just proximal to a thrombus or clot removed by the MT. In some embodiments, the method also involves obtaining a proximal sample comprising blood collected proximal to a thrombus in the subject, and a distal sample comprising blood collected distal to the thrombus in the subject. In some embodiments, the method also involves detecting cytokines and chemokines in the proximal sample and the distal sample, and predicting cerebral edema when there are increased amounts of cytokines and chemokines in the proximal sample as compared to the distal sample. In some embodiments, the method also involves detecting REG3A, CCL18, IL20RA, IL10RA, TNFRS9, ILS, KIT, TNF, CCL16, and GNLY proteins in the proximal sample and the distal sample, and predicting cerebral edema when there are increased amounts of REG3A, CCL18, IL20RA, IL10RA, TNFRS9, ILS, KIT, TNF, CCL16, and GNLY proteins in the proximal sample as compared to the distal sample.

In some embodiments of the presently-disclosed subject matter, when risk of cerebral edema in the subject has been identified, the method further involves administering or having administered a treatment to the subject. The administered treatment can be capable of mitigating cerebral edema.

In some embodiments, the treatment includes surgery, such as a decompressive craniectomy. In some embodiments, the treatment includes use of a therapeutic agent.

Examples of therapeutic agents that can be used in the treatment of cerebral edema include, for example, an osmolar agent, such as urea, mannitol, or hypertonic saline. Additional examples include diuretic, which can be beneficially used in combination with osmolar agents. Additional examples include anesthetics and sedatives.

Additional examples of therapeutic agents that can be used in the treatment of cerebral edema include, for example, curcuminoids, aquaporin inhibitors, such as AER-270 and AER-271, bumetanide, glibenclamide, SB-3CT, amiloride, SM-20220, HOE-642, SR 49059, VI880, conivaptan, vasopressin ML7, vascular endothelial growth factor (VEGF) inhibitors, such as bevacizumab, NK1 receptor antagonists, fenofibrate, pioglitazone, rosiglitazone, HMGB1, fingolimod, RPC1073, and RP101075. Additional examples are set forth in Shah (2016)³², and Halstead and Geocadin (2019)³³, which are incorporated herein by this reference in their entirety.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

As used herein, the term “subject” refers to a target of administration or medical procedure. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “demographics variables” refer to variables such as sex, age, race/ethnicity, BMI, comorbidities (hypertension, type 2 diabetes, hyperlipidemia, previous stroke), infarct volume, edema volume and the like.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Sample Acquisition

BACTRAC is a continually enrolling tissue registry. Other than pregnant or imprisoned individuals, all thrombectomy patients are considered for enrollment. Tissue samples were obtained and banked or processed in accordance with the published protocol.^{6,22} Briefly, at the beginning of the mechanical thrombectomy procedure, during initial access through the clot to the distal intracranial vessels, a microcatheter is navigated distal to the thrombus for backbleeding to determine if they are distal to the distal end of the thrombus. This allows for aspiration of 1 ml of whole arterial blood from the microcatheter (0.021 inch inner lumen diameter. Simultaneously, the collection of 7 ml of whole arterial peripheral blood was collected from the cervical parent artery (internal carotid artery or vertebral artery) during backbleeding while initiating suction for thrombectomy. The distal (intracranial) and proximal (peripheral, systemic) blood samples were immediately processed.

### Example 2: Sample Preparation

Samples are obtained on the first pass of thrombectomy to reduce any risk of cross-contamination of specimens. Plasma was immediately isolated and frozen at −80° C. Aliquots (40 μL) of plasma were randomized and placed into a 96-well plate (Starstedt, Nümbrecht, Germany), covered with Micro-Amp clear adhesion film (Thermo-Fisher Scientific), and shipped overnight on dry ice to Olink Proteomics (Olink Proteomics, Boston, Mass., USA). Standardized cardiometabolic and inflammatory panels were requested on 1 μL aliquots of plasma using a proximity extension assay (PEA). For this study, intracranial intraluminal blood distal to the thrombus was compared to each subject's systemic arterial blood, which provided an internal comparative control.

### Example 3: Protein Analysis

Olink Proteomics provides a Normalized Protein eXpression (NPX), a unit that is in log 2 scale which allows for individual protein analysis across a sample set. The intracranial versus systemic fold change in NPX was calculated by subtracting the systemic NPX (NPXₛ) from the intracranial NPX (NPXᵢ). In order to determine which proteins had the most significant changes, a series of 184 paired t-tests were performed, one for each of the 92 cardiometabolic and 92 inflammatory proteins. Within each panel, proteins were then ranked based on the associated p values. Benjamini and Hochberg's linear step up procedure was used to control the false discovery rate at 0.05.⁹ Additionally, linear regression was used to determine the impact of potential confounding factors on the degree to which protein expression differs in the intracranial versus systemic blood. For the top changing proteins, the original fold change was controlled for using baseline National Institutes of Health Stroke Scale (NIHSS) values. The significance of baseline NIHSS scores and the percent change in the original compared with adjusted fold change values were used to determine the impact of these potential confounders. All data analysis was performed using SAS software version 9.4 (SAS Institute Inc., Cary, N.C., USA).

### Example 4: Patient Characteristics

A medical record review was conducted to collect admission clinical and demographic variables. These variables included age, sex, ethnicity, height, weight, body mass index (BMI), comorbidities, smoking status, National Institutes of Health Stroke Scale (NIHSS), baseline computerized tomography (CT) and/or magnetic resonance imaging (MRI) scan to verify large vessel occlusion stroke/thrombectomy eligibility and CTA collateral score, and time of last known normal (LKN) symptom (prior to ischemic stroke). Additional relevant subject demographics and disease-related variables can include premorbid Modified Rankin Score (mRS), location of the thrombus (e.g. M1 segment of middle cerebral artery), likely source of the thrombus (cardioembolic, intracranial stenosis, dissection, carotid occlusion, infection, unknown), presence of tandem occlusion, time from Last Known Normal to thrombectomy completion (reopening of vessel), thrombectomy success as rated by TICI score, NIHSS on admission and prior to discharge, Mini-Montreal Cognitive Assessment prior to discharge, medical co-morbidities including hypertension, diabetes and hypercholesterolemia, pre-morbid modified Rankin Scale, and administration of intravenous tPA

Subject demographics are shown in Table 1. Twenty-five adult subjects were included in the study with a median age of 64 (24-91), of whom 15 (60%) were female. Ten subjects had a normal body mass index, 12 were overweight, and three were obese. Of note, 7 (28%) were current smokers, and 3 (12%) were previous smokers. According to the NIHSS score on admission, 1 (4%) of the patients had a minor stroke (NIHSS score 1-4), 9 (36%) were considered to have a moderate stroke (NIHSS score 5-15), and 8 (32%) were considered to have a moderate/severe stroke (NIHSS score 16-20). On discharge, 10 (40%) were considered to have a minor stroke, 11 (44%) were considered to have a moderate stroke, and 1 (4%) was considered to have a severe stroke (NIHSS>21). The mean last known normal to thrombectomy completion time was 513±246 min and the mean infarct volume was 58 172±82 284 mm³.

TABLE 1
Demographics and characteristics for thrombectomy subjects

| | Value (%) |
|---|---|
| Age (median; range) | 64 (24-91) |
| Sex | |
| Female | 15 (60) |
| Male | 10 (40) |
| BMI | |
| <24.9 | 10 (40) |
| 25-29.9 | 12 (48) |
| 30-39.9 | 3 (12) |
| >40 | 0 (0) |
| Comorbidities | |
| Hypertension | 16 (64) |
| Diabetes mellitus 2 | 4 (16) |
| Hyperlipidemia | 4 (16) |
| Previous stroke | 6 (24) |
| Previous myocardial infarction (MI) | 1 (4) |
| Smoking status | |
| Never | 15 (60) |
| Currently | 7 (28) |
| Previously (>6 months) | 3 (12) |
| NIHSS score on admission | |
| Minor stroke (1-4) | 1 (4) |
| Moderate stroke (5-15) | 9 (36) |
| Moderate/severe (16-20) | 8 (32) |
| Severe stroke (>21) | 7 (28) |
| NIHSS score at discharge* | |
| Minor stroke (1-4) | 10 (40) |
| Moderate stroke (5-15) | 12 (44) |
| Moderate/severe (16-20) | 0 (0) |
| Severe stroke (>21) | 2 (4) |
| TICI score | |
| 2a = <50% perfusion | 0 (0) |
| 2b = >50% perfusion | 10 (40) |
| 3 = full perfusion | 15 (60) |
| LKN to thrombectomy | |
| Completion time (min)* | 513 ± 246 |
| Infarct volume (mm³)† | 58 172 ± 82 284 |
| CTA collateral score† | |
| 0 | 4 (16) |
| 1 | 16 (64) |
| 2 | 3 (12) |

Values are median with range, mean ± SD, or (%).
*Data for one patient were missing (n = 24).
†Data for two patients were missing (n = 23).
BMI, body mass index; CTA, CT angiography; LKN, last known normal; MI, myocardial infarction; NIHSS, National Institutes of Health Stroke Scale; TICI, Thrombolysis in Cerebral Infarction.

### Example 5: Proteomics

The cardiometabolic panel analyzed 92 protein biomarkers, including proteins involved in cellular metabolic processes, cell adhesion, immune response, and complement activation. The inflammatory panel was an immune assay of 92 inflammation-related protein biomarkers. After controlling for the false discovery rate across all 184 proteins, 42 cardiometabolic and 53 inflammatory proteins had significantly different expressions in intracranial versus systemic blood. All these 95 proteins exhibited lower expression in the intra-cranial blood.

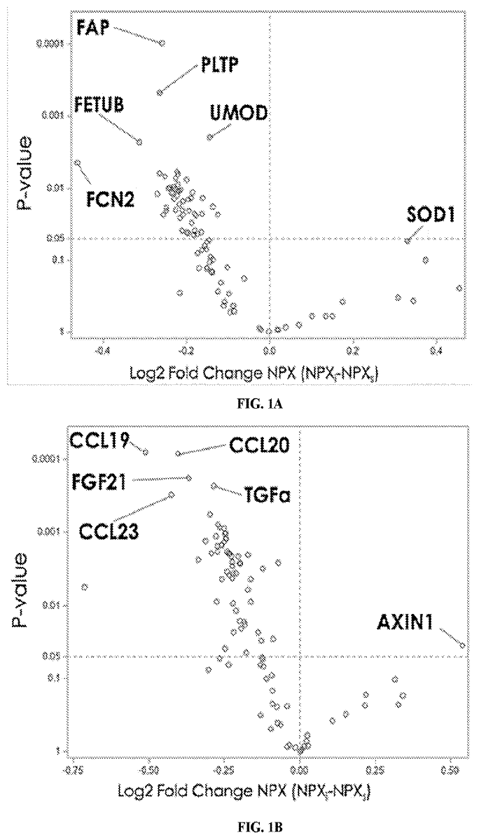

In FIGS. 1A and 1B, values were reported as the average changes in protein expression in distal plasma compared to proximal plasma. This was calculated by subtracting the proximal Normalized Protein Expression (NPX) from the distal NPX, where NPX is an arbitrary unit on a log 2 scale (NPXᵢ-NPXₛ). Thus, a difference of minus one implies the protein in question was twice as concentrated in the distal sample, and a difference of one implies the protein was twice as concentrated in the proximal sample.

A cardiometabolic panel volcano plot illustrating proteomic log 2 fold changes in Normalized Protein eXpression (NPX) in intracranial blood compared with systemic blood is provided in FIG. 1A. The top five-fold changes were: prolyl endopeptidase (FAP) at −0.26 (p′z0.0001), phospholipid transfer protein (PLTP) at −0.26 (p=0.0005), uromodulin (UMOD) at −0.14 (p=0.002), fetuin-B (FETUB) at −0.31 (p=0.002), and ficolin-2 (FCN2) at −0.46 (p=0.005). Proteins with negative fold change are located to the left of the vertical line and indicate higher expression in systemic blood. Proteins located above the horizontal line are significant (p<0.05).

An inflammatory panel volcano plot illustrating proteomic log 2 fold changes in Normalized Protein eXpression (NPX) in intracranial blood compared with systemic blood is provided in FIG. 1B. The top five-fold changes were: C—C motif chemokine 19 (CCL19) at −0.51 (p′z0.0001), C—C motif chemokine 20 (CCL20) at −0.40 (p′z0.0001), fibroblast growth factor 21 (FGF21) at −0.37 (p=0.0002), transforming growth factor alpha (TGF-α) at −0.28 (p=0.0002), and C—C motif chemokine (CCL23) at −0.43 (p=0.0003). Proteins with negative fold change are located to the left of the vertical line and indicate higher expression in systemic blood. Proteins located above the horizontal line are significant (p<0.05). Changes in AXIN1 were found to be non-significant after controlling for the false discovery rate. Although not statistically significant after controlling for the false discovery rate, the two proteins whose fold change in expression had the greatest increase in the intracranial blood were axin-1 (AXIN1) at 0.54 (p=0.035) and superoxide dismutase 1 (SOD1) at 0.33 (p=0.055) from the inflammatory and cardiometabolic panels, respectively.

### Example 6: Linear Regression Analyses

Table 2 presents results from the series of linear regression analyses predicting the fold changes from the 12 proteins labeled in the volcano plots (FIGS. 1A and 1B) controlling for baseline NIHSS values. All adjusted fold change values were significant. For the six cardiometabolic proteins, controlling for stroke severity resulted in adjusted fold change values that were nearly identical to the originals (largest percent change=0%; smallest p value for NIHSS score predicting fold change=0.3955).

For the top five inflammatory proteins with higher expression in the systemic blood, controlling for baseline NIHSS score resulted in an average adjustment of −15.06%. However, baseline NIHSS score was only significantly related to the fold change in TGF-α (p=0.025). Higher levels of baseline NIHSS values were associated with larger discrepancies in protein expression in intracranial blood compared with systemic blood.

TABLE 2
Impact of stroke severity on difference in protein
expression between intracranial and systemic blood

| | | | | | | NIHSS score | |
|---|---|---|---|---|---|---|---|
| | Original | | Adjusted | | | at admission | |
| | fold change | | fold change | | | significance | |
| | NPXᵢ- | P | NPXᵢ- | % | P | Slope | P |
| Protein panel | NPXₛ | value | NPXₛ | Change* | value | estimate | value |
| Cardiometabolic | | | | | | | |
| FAP | −0.2604 | <0.0001 | −0.2604 | 0.00% | 0.0001 | −0.0072 | 0.3955 |
| PLTP | −0.2647 | 0.0005 | −0.2647 | 0.00% | 0.0006 | 0.0012 | 0.9018 |
| FETUB | −0.3136 | 0.0023 | −0.3136 | 0.00% | 0.0028 | −0.0034 | 0.8104 |
| UMOD | −0.1437 | 0.0020 | −0.1437 | 0.00% | 0.0024 | −0.0030 | 0.6362 |
| FCN2 | −0.4628 | 0.0045 | −0.4628 | 0.00% | 0.0053 | 0.0076 | 0.7375 |
| SOD1 | 0.3290 | 0.0548 | 0.3290 | 0.00% | 0.0601 | −0.0043 | 0.8631 |
| Inflammatory | | | | | | | |
| CCL19 | −0.5111 | <0.0001 | −0.4358 | −14.73% | <0.0001 | −0.0186 | 0.1261 |
| CCL20 | −0.4034 | <0.0001 | −0.3483 | −13.66% | <0.0001 | −0.0137 | 0.1839 |
| FGF21 | −0.3687 | 0.0002 | −0.3114 | −15.54% | <0.0001 | −0.0122 | 0.2035 |
| TGFα | −0.2839 | 0.0002 | −0.2422 | −14.69% | <0.0001 | −0.0173 | 0.0250 |
| CCL23 | −0.4255 | 0.0003 | −0.3546 | −16.66% | <0.0001 | −0.0155 | 0.1730 |
| AXIN1 | 0.5389 | 0.0349 | 0.5611 | 4.12% | 0.0382 | 0.0130 | 0.7345 |

*(Original fold change − adjusted fold change)/original fold change.
AXIN1, axin-1;
CCL, C—C motif chemokine;
FAP, prolyl endopeptidase;
FCN2, ficolin-2;
FETUB, fetuin-B;
FGF21, fibroblast growth factor 21;
PLTP, phospholipid transfer protein;
SOD1, superoxide dismutase 1;
TGFα, transforming growth factor alpha;
UMOD, uromodulin

### Example 7: Infarct Volume and Edema Volume Calculation

Infarct and edema volumes were calculated using 24-hour post-thrombectomy MRI. Diffusion weighted images (DWI) were used to calculate infarct volumes, and T₂ FLAIR images were used to calculate edema volumes. The areas of abnormal signal (restricted diffusion on DWI or hyperintense signal on T₂ FLAIR) were manually segmented and analyzed by using ITK-SNAP software (www.itksnap.org).²⁷ All imaging assessment was performed by an experienced neuroradiologist in a blinded fashion.

### Example 8: Infarct Volume and Edema Volume Prediction

Machine learning (ML) is used to allow for the integration of several types of data including imaging, genetic, proteomic, and clinical/experimental analyses into a single algorithm. Rather than analyzing each biomarker individually, ML is used to identify and analyze clusters of biomarkers. By integrating multiple levels of data, this ML model allows for better prediction of clinical outcomes after stroke. The analysis disclosed herein used ML to detect plasma proteins that are predictors of infarct volume and edema in stroke patients. The algorithm found that proteins CCL18, IL10Ra, IL20Ra and REG3A are predictors for both edema and infarct volume.

For ML analysis, the demographics data used two stroke outcomes (infarct and edema volumes) along with the aforementioned plasma protein dataset. Prognosis of the stroke outcomes of edema and infarct volumes on the plasma expression dataset were performed with machine learning methods, Feature and Lasso, making use of the predictive protein panel discovered and disclosed herein.

First, the edema volume was taken as an outcome and proteins were measured as different features. A subset of most relevant features was identified and used create an algorithm for prognosticating the stroke outcome accurately and reliably. Since of the sample size of the plasma data and the number of features (24 subjects with 184×2 features), the discovery and prognosis needed to handle the high-dimensional setting. Also, the protein expression levels measured from distal (D) and proximal (P) blood were correlated.

A known ML method, extremely randomized trees (ERT),²⁸ was leveraged for pinpointing the biosignature to prognosticate the outcome. This method was adopted because of its known learning capabilities of handling small-sample data and correlations, which are potentially non-linear, between many features. The method was used to weigh the importance of features for selection and also to carry out regression on the selected features.

The developed model allows for predicting outcomes for new examples that were unseen in the training data. Specifically, extremely randomized trees (ERT) was performed 100 times with different random seeds and the mean values of the feature-importances were obtained. The number of possible orders of the features is combinatorically large, but this approach can effectively reduce the computational complexity and the effect of correlations between features.

Out of the 184×2 plasma proteins, 10 were discovered to be significant predictors of infarct volume as ranked by their mean importance values by (Table 3). Similar to the identification of a subset of markers and prediction of infarct volume, the predictive analysis of edema was performed by taking it as an output and all the measured plasma proteins as features. Four of these overlapped with edema volume prediction (Table 3).

Table 3 includes significant ML data for infarct volume prediction with overlap in edema volume denoted by bolded and underlined text. “Feature” column shows the top features identified by ERT, and “Lasso” by Lasso.

TABLE 3
Infarct V olume Prediction
Infarct Overlap

| Feature | | Lasso | |
|---|---|---|---|
| REG3A-P | | | REG3A-D |
| | GNLY-D | GNLY-P | |
| CCL18-P | CCL18-D | CCL18-P | |
| IL5-P | | IL5-P | |
| KIT-P | | KIT-P | |
| TNF-P | | TNF-P | |
| CCL16-P | | CCL16-P | |
| IL20RA-P | | | IL20RA-D |
| TNFR59-P | | TNFR59-P | |
| IL10RA-P | | IL10RA-P | |

While ERT was tapped for its ability to handle small-sample, high-dimensional data, overfitting nonetheless occurred which affected the generalizability of the model and lowered the testing accuracy. To ameliorate or even eliminate this issue, several other methods were explored, which are potentially applicable in small-sample, high-dimensional settings, including Lasso,²⁷ random forest (RF) regression,^{28,29} and support vector regression (SVR).

Lasso uses a linear combination of the features for predictive modeling. Notably, it incorporates a sparsity-inducing regularizer in its objective function to control the model complexity, and thus it may exploit the sparsity for selecting a subset of features to yield improved generalization by the structural risk minimization principle²⁷. In the experiments, the 5-fold cross-validation predictive error was used as an estimate of the generalization error. It was found that Lasso exhibited a higher prognosis accuracy than the other ML methods on this protein dataset, likely because there is an underlying sparse subset of features for edema and infarct volume. Also, with a small sample size the potential nonlinear effect of the features could not be captured sufficiently while a linear model like Lasso may suffice.

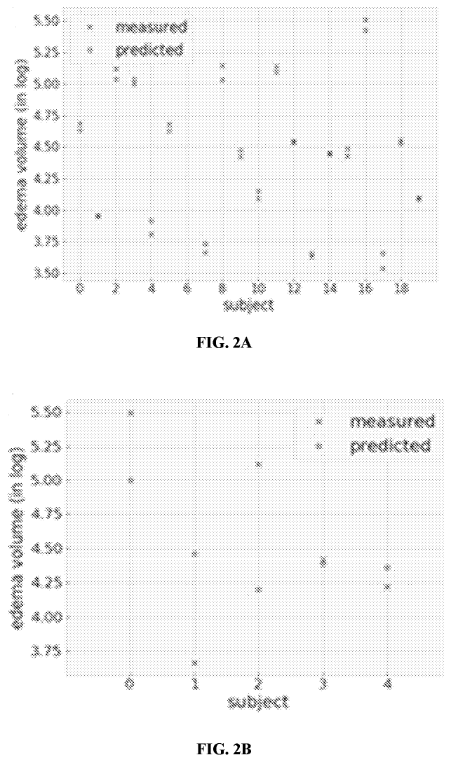

With reference to FIGS. 2A and 2B, the predicted and measured edema values were obtained by Lasso on the training data (FIG. 2A) and testing data (FIG. 2B) with a ratio of 4:1 random split during a 5-fold cross validation. It is contemplated that the inclusion of potentially critical confounding variables, such as gender, ethnicity, and age, as candidate features would further boost the predictive performance. By using Lasso on the subset of features selected by ERT, further improvement in prognostic accuracy could be made, as revealed with the 5-fold cross validation results. It indicated the existence of a nonlinear relationship between features for predicting stroke outcomes.

Resulting from this data, a program is contemplated for using an algorithm to predict stroke outcomes based on the expression of the protein plasma biomarkers occurring during stroke. This prognostic technology is useful to promote personalized medicine for stroke patients.

### Example 9: Proteomic Changes at the Site of Infarct

Disclosed herein are proteomic changes discovered to be occurring at the site of infarct in ischemic stroke in the human condition. Data provided herein offer insight into biomolecular and cell signaling responses that occur hours after vessel occlusion.

Of the cardiometabolic proteins, SOD1 is an antioxidant that is known to protect from reactive oxygen species after cerebral ischemia and reperfusion. Further, in mouse models, it has been shown that overexpression of SOD1 prevents the early release of mitochondrial cytochrome c after focal ischemia and reper-fusion.¹⁰ These protective effects prove to be particularly interesting in these data, as SOD1 showed a trend (p=0.055) of higher expression in the intracranial blood and may be contributing to a neuroprotective response.

The protein PLTP is present in human platelets and has been shown to play a role in the initiation of thrombin generation and platelet aggregation participating in hypercoagulation.¹¹ PLTP has also been shown to have hyperlipidemic properties and that increased expression can increase the risk of cardiovascular disease in humans.12¹³ Additionally, FCN2 has been shown to be deposited in human carotid plaques and may play a role in complement activation and atherosclerosis.¹⁴

The protein FETUB has been reported to be elevated in patients with acute myocardial infarction in comparison with patients with stable angina.¹⁵ This study suggests that FETUB may play a role in acute myocardial infarction modulation, lipid deposition, and plaque-stabilizing factors in regards to ischemia. The roles of PLTP, FCN2, and FETUB in stroke predisposition and outcome and stratified based on factors such as coagulopathies, anticoagulation/antiplatelet medications, and dyslipidemia, are of interest.

Studies have reported that neuropeptides such as FAP play a role in central nervous system neurotransmission and neuromodulation. Specifically, administration of an FAP inhibitor may work to ameliorate memory impairment due to middle cerebral artery occlusion by restoring the decreased thyrotropin-releasing hormone activity in rodents.¹⁶ However, the literature is underdeveloped with regard to the relationship of FAP with acute ischemic stroke in humans and may provide an opportunity for these data to establish a novel relationship between FAP and ELVO stroke. Additionally, UMOD is known to be expressed in the thick ascending loop of the kidneys, and no clear relationship with ischemic stroke has been previously reported.

Of the inflammatory proteins, it has been reported that FGF21 may play a neuroprotective role against injury in cerebral micro-vascular endothelial cells during hypoxic stress.¹⁷ As this protein was found to have significantly lower expression levels in the intracranial samples, it may indicate a diminished or slower local neuroprotective response or extravasation of FGF21 into brain parenchyma. It is contemplated that FGF21 might relate to infarct time and infarct volume.

A complex set of chemokine signaling occurring at the time of infarct is reported herein. CCL23 is known to be a chemotactic agent, probably involved in inflammation and atherosclerosis.¹⁸ CCL23 blood levels can discriminate brain damaging diseases and may be a biomarker of stroke prognosis and predictor of patient outcome at hospital discharge.¹⁹ The same study found that patients with acute cerebral injury present with higher baseline levels of circulating CCL23. This response may be more systemic rather than local, as our study demonstrates lower expression levels of CCL23 at the site of infarct than systemically. Future studies using age-matched controls will help establish timeline and locality of this chemokine response. The interaction of CCL19 and its receptor CCR7 may play an important role in arteriogenesis in ischemia, as well as the migration and homing of T-lymphocytes.²⁰ Reduced intracranial levels of CCL23 and CCL19 may be due to chemokine binding, chemokine activation timeline, or extravasation out of the intraluminal space.

As reported herein, stroke severity, based on baseline NIHSS score, relates to the discrepancy of expression levels between intracranial and systemic arterial blood samples. This finding may be due to diminished peri-infarct levels of TGF-α, leading to a dampened neuroprotective effect. The role TGF-α plays in stroke severity, infarct volume, and functional recovery is of interest.

It was previously reported that genes related to a Th2 autoimmune response are elevated in intracranial blood from subjects during thrombectomy.⁸ Changes in CCL23 support the gene data as CCL23 is induced by interleukin 4, a classic Th2 cyto-kine.²² Proteins are drug targets for ischemic stroke.²³ Th2 drug targets already exist and have been developed to alter pathways for conditions such as asthma.²⁴ Understanding these responses occurring at the site of infarct in ischemic stroke are useful in connection with drug development and particularly, in the area of repurposing FDA approved agents for use as an adjuvant for the thrombectomy procedure.

Interestingly, the significant proteins reported here were found to have a lower level of expression in the intracranial (distal) blood than in systemic (proximal) blood. This finding may be due to a combination of factors. It may indicate proteomic extravasation into brain parenchyma as many of these proteins are inflammatory, reactive, and migratory. It may be a result of the protein expression timeline and how that relates to infarct time for each subject with stroke. Other possible explanations include cellular binding of proteins preventing extraction from plasma, low levels of collateral blood supply precluding protein influx into the intra-luminal sample space, or a robust systemic response blunting our primary measure, change in NPX. Lastly, this finding may be related to the cohort of patients with varying stroke severity, baseline characteristics, and stroke etiology.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Benjamin E J, Muntner P, Alonso A, et al. Heart disease and stroke statistics-2019 update: a report from the American Heart Association. Circulation 2019; 139:e56-28.
2. Campbell B C V, Mitchell P J, EXTEND-IA Investigators. Endovascular therapy for ischemic stroke. N Engl J Med 2015; 372:2365-6.
3. Fransen P S S, Beumer D, Berkhemer O A, et al. Mr CLEAN, a multicenter randomized clinical trial of endovascular treatment for acute ischemic stroke in the Netherlands: study protocol for a randomized controlled trial. Trials 2014; 15:343.
4. Goyal M, Demchuk A M, Menon B K, et al. Randomized assessment of rapid endovascular treatment of ischemic stroke. N Engl J Med 2015; 372:1019-30.
5. Saver J L, Goyal M, Bonafe A, et al. Solitaire™ with the intention for thrombectomy as primary endovascular treatment for acute ischemic stroke (SWIFT PRIME) trial: protocol for a randomized, controlled, multicenter study comparing the Solitaire revascularization device with IV tPA with IV tPA alone in acute ischemic stroke. Int J Stroke 2015; 10:439-48.
6. Fraser J F, Collier L A, Gorman A A, et al. The blood and clot thrombectomy registry and collaboration (BACTRAC) protocol: novel method for evaluating human stroke. J Neurointerv Surg 2019; 11:265-270.
7. Martha S R, Fraser J F, Pennypacker K R. Acid-base and electrolyte changes drive early pathology in ischemic stroke. Neuromolecular Med 2019; 21:540-5.
8. Martha S R, Cheng Q, Fraser J F, et al. Expression of cytokines and chemokines as predictors of stroke outcomes in acute ischemic stroke. Front Neurol 2019; 10:1391.
9. Benjamini Y, Hochberg Y. Controlling the false discovery rate: a practical and powerful approach to multiple test. Journal of the Royal Statistical Society 1995; 57:289-300.
10. Fujimura M, Morita-Fujimura Y, Noshita N, et al. The cytosolic antioxidant copper/zinc-superoxide dismutase prevents the early release of mitochondrial cytochrome c in ischemic brain after transient focal cerebral ischemia in mice. J Neurosci 2000; 20:2817-24.
11. Zhao X-M, Wang Y, Yu Y, et al. Plasma phospholipid transfer protein promotes platelet aggregation. Thromb Haemost 2018; 118:2086-97.
12. Desrumaux C, Deckert V, Lemaire-Ewing S, et al. Plasma phospholipid transfer protein deficiency in mice is associated with a reduced thrombotic response to acute intravascular oxidative stress. Arterioscler Thromb Vasc Biol 2010; 30:2452-7.
13. Tzotzas T, Desrumaux C, Lagrost L. Plasma phospholipid transfer protein (PLTP): review of an emerging cardiometabolic risk factor. Obes Rev 2009; 10:403-11.
14. Pilely K, Rosbjerg A, Genster N, et al. Cholesterol crystals activate the lectin complement pathway via ficolin-2 and mannose-binding lectin: implications for the progression of atherosclerosis. J Immunol 2016; 196:5064-74.
15. Jung S H, Won K-J, Lee K P, et al. The serum protein fetuin-B is involved in the development of acute myocardial infarction. Clin Sci 2015; 129:27-38.
16. Shinoda M, Matsuo A, Toide K. Pharmacological studies of a novel prolyl endopeptidase inhibitor, JTP-4819, in rats with middle cerebral artery occlusion. Eur J Pharmacol 1996; 305:31-8.
17. Wang H-W, Jiang X, Zhang Y, et al. Fgf21 protects against hypoxia injury through inducing HSP72 in cerebral microvascular endothelial cells. Front Pharmacol 2019; 10:101.
18. Kim J, Kim Y S, Ko J. C K beta 8/CCL23 induces cell migration via the Gi/Go protein/PLC/PKC delta/NF-kappa B and is involved in inflammatory responses. Life Sci 2010; 86:300-8.
19. Simats A, Garcia-Berrocoso T, Penalba A, et al. Ccl23: a new CC chemokine involved in human brain damage. J Intern Med 2018; 283:461-75.
20. Nossent A Y, Bastiaansen A J N M, Peters E A B, et al. CCR7-CCL19/CCL21 axis is essential for effective arteriogenesis in a murine model of hindlimb ischemia. J Am Heart Assoc 2017; 6. doi:10.1161/JAHA.116.005281. [Epub ahead of print: 8 Mar. 2017].
21. Leker R R, Toth Z E, Shahar T, et al. Transforming growth factor alpha induces angiogenesis and neurogenesis following stroke. Neuroscience 2009; 163:233-43.
22. Novak H, Müller A, Harrer N, et al. Ccl23 expression is induced by IL-4 in a STAT6-dependent fashion. J Immunol 2007; 178:4335-41.
23. Chong M, Sjaarda J, Pigeyre M, et al. Novel drug targets for ischemic stroke identified through Mendelian randomization analysis of the blood proteome. Circulation 2019; 140:819-30.
24. Roufosse F. Targeting the interleukin-5 pathway for treatment of eosinophilic conditions other than asthma. Front Med 2018; 5:49.
25. Kollikowski A M, Schuhmann M K, Nieswandt B, et al. Local leukocyte invasion during hyperacute human ischemic stroke. Ann Neurol 2020; 87:466-79.
26. Simpkins A N, Janowski M, Oz H S, et al. Biomarker application for precision medicine in stroke. Transl Stroke Res 2019. doi:10.1007/s12975-019-00762-3. [Epub ahead of print: 18 Dec. 2019].
27. Gauvreau G M, FitzGerald J M, Boulet L P, et al. The effects of a CCR3 inhibitor, AXP1275, on allergen-induced airway responses in adults with mild-to-moderate atopic asthma. Clin Exp Allergy 2018; 48(4):445-51. doi: 10.1111/cea.13114 [published Online First: 2018 Feb. 10]
28. Yoshie O, Matsushima K. CCR4 and its ligands: from bench to bedside. Int Immunol 2015; 27(1):11-20. doi: 10.1093/intimm/dxu079 [published Online First: 2014 Aug. 5]
29. Kumai T, Nagato T, Kobayashi H, et al. CCL17 and CCL22/CCR4 signaling is a strong candidate for novel targeted therapy against nasal natural killer/T-cell lymphoma. Cancer Immunol Immunother 2015; 64(6):697-705. doi: 10.1007/s00262-015-1675-7 [published Online First: 2015 Mar. 11]
30. Butterfield J H, Leiferman K M, Abrams J, et al. Elevated serum levels of interleukin-5 in patients with the syndrome of episodic angioedema and eosinophilia. Blood 1992; 79(3):688-92. [published Online First: 1992 Feb. 1]
31. Tan S, Shan Y, Lin Y, et al. Neutralization of interleukin-9 ameliorates experimental stroke by repairing the blood-brain barrier via down-regulation of astrocyte-derived vascular endothelial growth factor-A. FASEB J 2019; 33(3):4376-87. doi: 10.1096/fj.201801595RR [published Online First: 2019 Jan. 30]
32. Shah S, Kimberly W T. Today's Approach to Treating Brain Swelling in the Neuro Intensive Care Unit. Semin Neurol. 2016; 36(6):502-507. doi:10.1055/s-0036-1592109
33. Halstead M R, Geocadin R G. The Medical Management of Cerebral Edema: Past, Present, and Future Therapies. Neurotherapeutics. 2019; 16(4):1133-1148. doi:10.1007/s13311-019-00779-4
34. Robert S M, Reeves B C, Alper S L, Zhang J, Kahle K T. New drugs on the horizon for cerebral edema: what's in the clinical development pipeline? Expert Opin Investig Drugs. 2020 October; 29(10):1099-1105. doi: 10.1080/13543784.2020.1813715. Epub 2020 Sep. 20. PMID: 32815401; PMCID: PMC8104020.
35. International Patent Application Publication No. WO 2021/072404 to Pennypacker, et al. for “A machine learning algorithm for predicting clinical outcomes and identifying drug targets in ischemic stroke.”
36. Martha S R, Cheng Q, Fraser J F, Gong L, Collier L A, Davis S M, Lukins D, Alhajeri A, Grupke S, Pennypacker K R. Expression of Cytokines and Chemokines as Predictors of Stroke Outcomes in Acute Ischemic Stroke. Front Neurol. 2020 Jan. 15; 10:1391. doi: 10.3389/fneur.2019.01391. PMID: 32010048; PMCID: PMC6974670.
37. Maglinger B, Frank J A, McLouth C J, Trout A L, Roberts J M, Grupke S, Turchan-Cholewo J, Stowe A M, Fraser J F, Pennypacker K R. Proteomic changes in intracranial blood during human ischemic stroke. J Neurointerv Surg. 2021 April; 13(4):395-399. doi: 10.1136/neurintsurg-2020-016118. Epub 2020 Jul. 8. PMID: 32641418; PMCID: PMC7982920.

It will be understood that various details of the presently disclosed subject matter can be changed without departing from the scope of the subject matter disclosed herein. Furthermore, the foregoing description is for the purpose of illustration only, and not for the purpose of limitation.

1. A method of identifying risk of cerebral edema in a subject, comprising:
a) obtaining a sample comprising systemic blood from the subject;
b) detecting REG3A, CCL18, IL20RA, and IL10RA proteins in the sample;
c) identifying risk of cerebral edema in the subject when REG3A, CCL18, IL20RA, and IL10RA are detected in the sample.

2. The method of claim 1, and further comprising isolating plasma from the sample for use in detecting the proteins.

3. The method of claim 1, wherein the subject is a stroke patient who has undergone mechanical thrombectomy (MT).

4. The method of claim 3, wherein the systemic blood sample is peripheral blood collected just proximal to a thrombus removed by the MT.

5. The method of claim 3, and further comprising obtaining a proximal sample comprising blood collected proximal to a thrombus in the subject, and a distal sample comprising blood collected distal to the thrombus in the subject.

6. The method of claim 5, and further comprising detecting REG3A, CCL18, IL20RA, and IL10RA proteins in the proximal sample and the distal sample, and predicting cerebral edema when there are increased amounts of REG3A, CCL18, IL20RA, and IL10RA proteins in the proximal sample as compared to the distal sample.

7. The method of claim 3, and further comprising isolating plasma from the sample for use in detecting the proteins.

8. The method of claim 1, and further comprising administering a treatment to the subject.

9. The method of claim 8, wherein the treatment is capable of mitigating cerebral edema.

10. The method of claim 9, wherein the treatment comprises a therapeutic agent.

11. The method of claim 10, wherein the therapeutic agent is selected from the group consisting of: an osmolar agent, a diuretic, an anesthetic, a sedative, and combinations thereof.

12. The method of claim 10, wherein the treatment comprises surgery

13. The method of claim 12, wherein the surgery is a decompressive craniectomy.

14. The method of claim 1, and further comprising detecting amounts of TNFRS9, ILS, KIT, TNF, CCL16, and GNLY in the samples, and predicting infarct volume when TNFRS9, ILS, KIT, TNF, CCL16, and GNLY are detected in the sample.

15. The method of claim 13, wherein the subject is a stroke patient who has undergone mechanical thrombectomy (MT).

16. The method of claim 15, wherein the systemic blood sample is peripheral blood collected just proximal to a thrombus removed by the MT.

17. The method of claim 15, and further comprising obtaining a proximal sample comprising blood collected proximal to a thrombus in the subject, and a distal sample comprising blood collected distal to the thrombus in the subject.

18. The method of claim 17, and further comprising detecting REG3A, CCL18, IL20RA, IL10RA, TNFRS9, ILS, KIT, TNF, CCL16, and GNLY proteins in the proximal sample and the distal sample, and predicting cerebral edema when there are increased amounts of REG3A, CCL18, IL20RA, IL10RA, TNFRS9, ILS, KIT, TNF, CCL16, and GNLY proteins in the proximal sample as compared to the distal sample.

19. The method of claim 14, and further comprising isolating plasma from the sample for use in detecting the proteins.

20. The method of claim 14, and further comprising using a device to detect the proteins in the sample, wherein the device comprises a combination of probes affixed to a substrate, comprising a probe specific for each of REG3A, CCL18, IL20RA, IL10RA, TNFRS9, ILS, KIT, TNF, CCL16, and GNLY.

21. The method of claim 1, and further comprising using a device to detect the proteins in the sample, wherein the device comprises a combination of probes affixed to a substrate, comprising a probe specific for each of REG3A, CCL18, IL20RA, and IL10RA.

22. A device for use in identifying risk of cerebral edema in a subject, comprising: a combination of probes affixed to a substrate, comprising a probe specific for each of REG3A, CCL18, IL20RA, and IL10RA.

23. The device of claim 22, and further comprises a probe specific for each of TNFRS9, ILS, KIT, TNF, CCL16, and GNLY.

24. The device of claim 22, provided as a microfluidic enzyme-linked immunosorbent assay (ELISA) device.
